(19) United States
(12) Patent Application Publication
(10) Pub. No.: US 2011/0303220 A1
(43) Pub. Date: Dec. 15, 2011

(54) BREATHING TUBE APPARATUS FOR FACIAL SKIN TREATMENT PROCEDURES

(75) Inventor: SALLY J. BRIDGES-SCHIFFER, SAWYER, MI (US)

(73) Assignee: BRIDGES TO BEAUTY, LLC, Sawyer, MI (US)

(21) Appl. No.: 13/215,575

(22) Filed: Aug. 23, 2011

Related U.S. Application Data
(63) Continuation of application No. 11/701,702, filed on Feb. 2, 2007, now abandoned.

(51) Int. Cl. A61M 35/00; A62B 9/06

(52) U.S. Cl. 128/202.16

(57) ABSTRACT

A breathing apparatus for use in facial skin treatment procedures to allow a person being treated to breath without breathing through their nose or open mouth while substantially maintaining facial tissue exposed for application of a fluid treatment material including a breathing tube having one end for insertion into a person's mouth between the lips and teeth for breathing and a second end spaced from the first for access to air substantially uncontaminated by the fluid treatment material or by fumes of the fluid treatment material. A mouth piece on the one end of the breathing tube can have first and second radially extending engagement surfaces longitudinally spaced to receive the teeth of the person's upper and lower jaws between the first and second engagement surfaces when the mouthpiece is positioned in the person's substantially closed mouth.

### CROSS REFERENCE TO RELATED APPLICATIONS

This application is a continuation of prior application Ser. No. 11/701,702, filed Feb. 2, 2007, which application is hereby incorporated by reference.

### BACKGROUND OF THE INVENTION

(1) Field of the Invention

The invention relates to a breathing apparatus for use in connection with facial treatments that can include a wide variety of treatment materials that can be subtle, benign or potentially annoying and irritating. Such treatments can include chemical peels, Botox treatments and injections as well as other treatments and treatment materials.

(2) Description of Related Art

A variety of breathing tubes are known for a variety of uses including medical, snorkeling and emergency. Drinking and breathing tubes are also known.

### SUMMARY OF THE INVENTION

According to one aspect of the invention a breathing apparatus for use in facial skin treatment procedures to allow a person being treated to breath without breathing through their nose or open mouth while substantially maintaining facial tissue exposed for application of treatment material can have a breathing tube having one end for insertion into a person's mouth for breathing and a second end spaced from the first for access to air substantially uncontaminated by the treatment material, and can have a mouth piece on the one end of the breathing tube that can have an engagement surface arranged for contact with the teeth of at least one of the person's upper and lower jaws when the mouthpiece is positioned in the person's substantially closed mouth. The engagement surface can extend radially outward from the breathing tube.

According to another aspect of the invention a breathing apparatus for use in facial skin treatment procedures to allow a person being treated to breath without breathing through their nose or open mouth while substantially maintaining facial tissue exposed for application of treatment material can have a breathing tube having one end for insertion into a person's mouth for breathing and a second end spaced from the first for access to air substantially uncontaminated by the treatment material, and can have a mouth piece on the one end of the breathing tube that can have longitudinally spaced engagement surfaces for contact with the teeth of at least one of the person's upper and lower jaws when the mouthpiece is positioned in the person's substantially closed mouth.

There can be two engagement surfaces and one of the engagement surfaces can be arranged to be positioned between the lips and the outer surface of the teeth and the other engagement surface can be arranged to be positioned adjacent the inside surface of the teeth. The engagement surfaces can extend radially outward from the breathing tube for contact with the teeth of the person's upper and lower jaw.

The engagement surfaces can extend radially outward further in a direction along the jaws than in a direction toward the jaws of the person.

One of the engagement surfaces can have a substantially planar oval shape and can have the long and short axes in a plane generally perpendicular to the breathing tube. Both of the engagement surfaces can have a substantially planar oval shape and the long axes of the engagement surfaces can be substantially parallel.

According to one aspect of the invention the breathing tube can be rigid and can be between 6 and 12 inches long. Preferably the breathing tube can be between 8 and 10 inches long.

According to one aspect of the invention the mouthpiece can be molded of self supporting flexible plastic material. Alternately, the mouthpiece can be molded of soft deformable material.

According to one aspect of the invention the breathing apparatus can be a disposable, single use breathing apparatus and can be packaged in a sterile package.

According to another aspect of the invention a breathing apparatus for use in facial skin treatment procedures to allow a person being treated to breath without breathing through their nose or open mouth while substantially maintaining facial tissue exposed for application of a fluid treatment material can include a breathing tube having one end for insertion into a person's mouth between the lips and teeth for breathing and a second end spaced from the first for access to air substantially uncontaminated by the fluid treatment material or by fumes of the fluid treatment material, and can have a mouth piece on the one end of the breathing tube that can have first and second radially extending engagement surfaces longitudinally spaced to receive the teeth of the person's upper and lower jaws between the first and second engagement surfaces when the mouthpiece is positioned in the person's substantially closed mouth. The first engagement surface can be arranged to be positioned between the lips and the outer surface of the teeth and the second engagement surface can be arranged to be positioned adjacent the inside surface of the teeth.

The mouthpiece and breathing tube can be integrally molded of plastic material. Alternately, the mouthpiece and breathing tube can be separately molded of plastic material and assembled into a breathing apparatus.

### BRIEF DESCTRIPTION OF THE DRAWINGS

FIG. 1 is a partial schematic view of a patient on a reclining facial bed illustrating the use of a breathing apparatus according to the invention.

FIG. 2 is a profile view of a human head in a reclining position for a facial treatment showing the upper and lower jaw, teeth and upper and lower lips engaging a breathing apparatus according to the invention.

FIG. 3 is a perspective view of one embodiment of a breathing apparatus according to the invention.

FIG. 3A is a section view through the mouthpiece and a portion of the breathing tube of the breathing apparatus illustrated in FIG. 3.

FIG. 4. is a perspective view of another embodiment of a breathing apparatus according to the invention.

FIG. 4A is a section view through the mouthpiece and a portion of the breathing tube of the breathing apparatus illustrated in FIG. 3.

FIG. 5 is a perspective view of another embodiment of a breathing apparatus according to the invention.

FIG. 5A is a section view through the mouthpiece and a portion of the breathing tube of the breathing apparatus illustrated in FIG. 5.

FIG. 6 is an exploded view of a two piece embodiment of the breathing apparatus illustrated in FIG. 3.

FIG. 7 is a sectional view of a one piece embodiment of the breathing apparatus illustrated in FIG. 3.

FIG. 8 is an enlarged sectional view of a portion of FIG. 2 showing the upper and lower jaw, the upper and lower jaw teeth, upper and lower lips and a breathing apparatus according to the invention in greater detail.

FIG. 9 is a perspective view of a breathing apparatus according to the invention in a sterile package

### DESCRIPTION OF THE INVENTION

No one knows when or where humanity first discovered the tactile, sensual, cleansing and aesthetic benefits of a hot mud facial treatment. Age-old thermal, mineral and mud treatments have been passed down from many places and peoples around the globe. Today women and men have the opportunity to undergo a wide variety of beauty-enhancing treatments, from a hot mud facial to Botox injections to plastic surgery. One of the more popular contemporary facial treatments is known as a chem peel or chemical peel. Chemical peel treatments involve the application of a chemical solution to improve and smooth the texture of the facial skin by removing damaged outer layers. Chemical peel treatments can be helpful for individuals with facial blemishes, wrinkles and uneven skin pigmentation. Chemicals used for chemical peel treatments can include, but are not limited to, phenols, glycolic acid, trichloroacetic acid (TCA), salicylic acid, alphahydroxy acids (AHAs) ascorbic acid, kojic acid, betahydroxy acid (BHA), Obaji chemical peels and Jessner's peel. The chemical used and precise formulas are well known to those skilled in the art and can be selected and adjusted to meet each patient's needs and condition. Those skilled in the art will understand that palmitate forms of the chemicals can be used instead of natural acid form if desired. In this application facial treatment materials will sometimes be referred to as a treatment material or as a fluid treatment material. Applicant intends that treatment material and fluid treatment material should be understood to mean fluids, liquids, gels, muds, solutions and slurry materials and that the breathing apparatus according to the invention is intended to be used with any facial treatment material, without limitation. The breathing apparatus according to the invention can also be used with other facial treatment processes in which a patient's skin surface must be exposed for treatment and the patient might be more comfortable breathing through a breathing apparatus according to the invention instead of through his/her open mouth or nose.

A major drawback to receiving a chemical peel treatment can be the idea of having one's face treated with a chemical, particularly since the nose and mouth are parts of the face that are used for breathing. Thus, a person being treated does not have much choice but to inhale the chemical fumes if the person is unable to hold his or her breath for the duration of the treatment. For some persons breathing the chemical fumes may be merely uncomfortable, but for persons with asthma or other respiratory problems, or for pregnant women the discomfort may be severe or even hazardous. One solution has been to direct a ventilation fan at a patient's face, however, air flow from a fan can cause the treatment material to dry out too fast and diminish the efficacy of the treatment. A breathing apparatus according to the invention can alleviate a person's need to breath through their nose or open mouth during a facial treatment of any kind and possibly inhaling or ingesting treatment materials, byproducts of the treatment, or fumes of treatment materials.

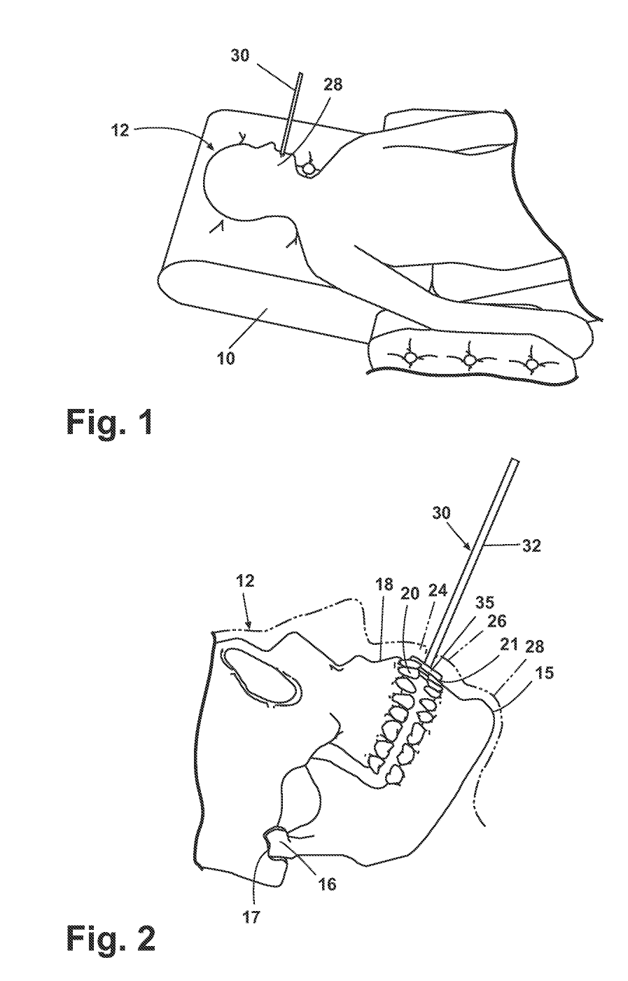

As may be seen in FIG. 1, a patient 12 can be seen in schematic form on a typical reclining facial bed or chair 10 as are well known in the art. A breathing apparatus 30 is shown projecting from the patient's mouth as will be described in detail below with the patient's face surface 28 exposed and ready for treatment by a skin care professional, not shown.

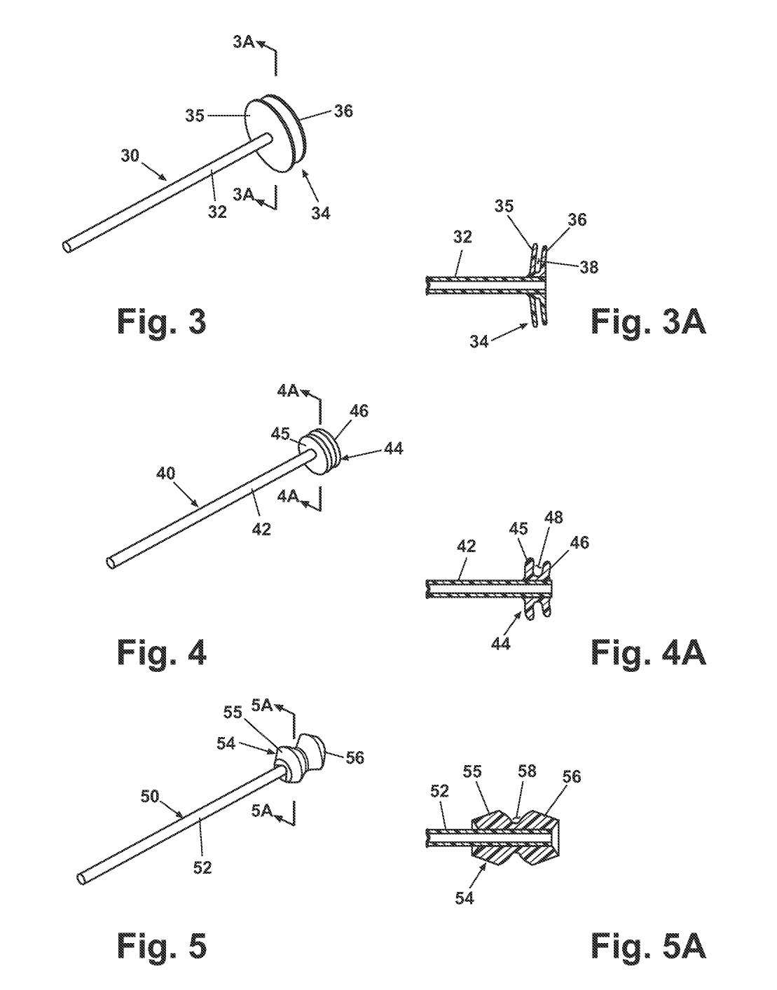

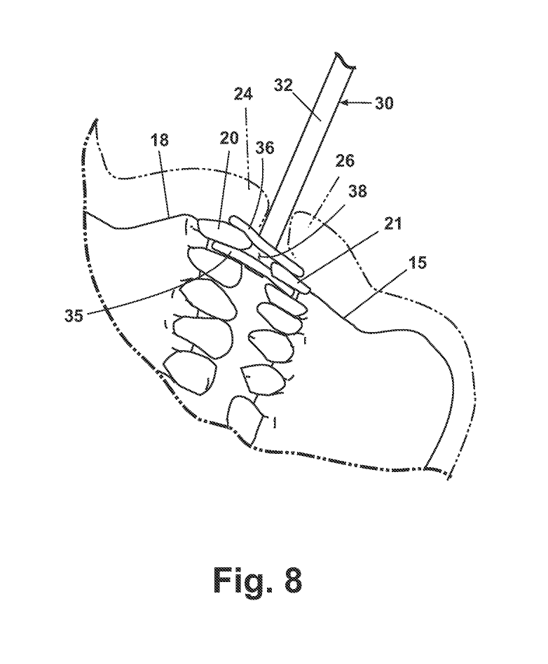

As may be seen in FIG. 2 a lower jaw bone 15 has a ball joint 16 that fits into a socket 17 of the upper jaw bone 18 and is held there in a way permitting movement of the lower jaw bone 15 relative to the socket as held and controlled by muscular structure, not shown. As may be seen in FIG. 2 and FIG. 8, a breathing apparatus 30 can be positioned in the mouth of a person between upper jaw teeth 20 and lower jaw teeth 21 and between upper lip 24 and lower lip 26. Breathing apparatus 30 can include a breathing tube 32 that can extend from mouthpiece 34 (see FIG. 3) to allow the patient to breath air that is not contaminated by treatment material, not shown, on the surface 28 of the patient's face. As may be seen in FIG. 3 and FIG. 3A, breathing apparatus 30 can have a mouthpiece 34 positioned on one end of breathing tube 32. Breathing tube 32 can be a plastic tube formed of materials suitable for human contact and that are resistant to the treatment materials as are well known in the art. While breathing tube 32 can be flexible, it is preferable that breathing tube 32 be sufficiently rigid that a patient will not readily collapse the end of breathing tube 32 cutting off the flow of fresh air during the facial treatment that could be the case with a drinking straw. Breathing tube 32 should have a sufficient diameter to allow free breathing during the treatment process, but should be small enough to be held comfortably in a person's mouth. For example, breathing tube can be approximately ¼″ to ½″ in diameter. Breathing tube 32 should be long enough to extend outward from a patient's face beyond the treatment materials, and beyond any strata of treatment material/chemical fumes/byproducts of treatment above/in front of the patient's face to a zone of clear air. Typically, a breathing apparatus 30 can be six to twelve inches (6″-12″) long, preferably eight to ten inches (8″-10″) long. Those skilled in the art will appreciate that the diameter and/or the length of a breathing tube 32 for a breathing apparatus 30 can be larger or smaller and longer or shorter than the examples provided above depending on the nature of the treatment material and how far from a patient's face fumes from the treatment material are likely to extend. Likewise, while the breathing tube 32 is illustrated as a straight tube, breathing tube 32 can be curved or bendable within the scope of the invention.

Mouthpiece 34 can have a first engagement surface 35 that can be arranged to be positioned between upper and lower jaw teeth 20, 21 and upper and lower lips 24, 26. Mouthpiece 34 can also have a second engagement surface 36 that can be positioned to be inside the upper and lower jaw teeth 20, 21 when the breathing apparatus 30 is positioned in a mouth. While the mouthpiece illustrated in FIG. 3 and FIG. 3A includes first and second engagement surfaces 35, 36 that extend radially outwardly from breathing tube 32 in all directions, the first and second engagement surfaces could be configured to extend outwardly in one direction to engage only the teeth 20, 21 in the upper or lower jaw 18, 15. Engagement surfaces 35, 36 serve to engage the teeth 20 of upper jaw 18 and/or teeth 21 of the lower jaw 15 and either upper lip 24 and/or lower lip 26 to assure that breathing tube 32 does not slide further into, or out of a patient's mouth during the facial treatment process. Thus, mouthpiece 34 can assure that breathing apparatus 30 will remain in a patient's mouth during a facial treatment process even in the event that the patient's lips become numb as can occur with Botox and other chemical treatments, or the patient becomes nervous or anxious during the treatment procedure. Thus, mouthpiece 34 can engage the teeth 20, 21 and lips 24, 26 of a patient to substantially prevent the patient from either ingesting or losing a breathing apparatus as could occur if a simple tube without a mouthpiece according to the invention is used for a breathing apparatus.

Mouthpiece 34 can be molded of plastic or synthetic rubber material, or if desired, wax or well known oral insert or mouthpiece materials. As noted above, breathing tube 32 can be molded of plastic of synthetic rubber material. Preferably, breathing tube 32 should be sufficiently rigid that breathing tube 32 will not readily collapse if a patient bites down on breathing tube 32 during the course of the treatment. Breathing tube 32 is illustrated in the drawings as a generally round in cross section. Breathing tube 32 can oval or generally rectangular in cross section shape having rounded ends within the scope of the invention as the specific cross sectional shape of breathing tube 32 can be other than round if desired. Breathing tube 32 and mouthpiece 34 can be independently formed and assembled or, can be integrally molded as one piece.

Referring to FIG. 3 and FIG. 3A, engagement surfaces 35, 36 can be generally oval in shape with the long axis extending along the jaw so that the engagement surfaces 35, 36 can generally overlay two to four teeth along the upper and lower jaws 18, 15. Engagement surfaces 35, 36 can have a short axis extending toward the upper and lower jaws 18, 15 so that engagement surfaces 35, 36 can overlay teeth 20, 21 in the upper and lower jaws 18, 15. Thus, engagement surfaces 35, 36 can assist in sealing the breathing apparatus 30 to a patient's mouth, particularly if a patient becomes anxious or if a patient's lips become numb from the treatment material, and help assure that a patient will breathe uncontaminated air as the facial treatment procedure proceeds. As noted above, engagement surfaces 35, 36 should be sufficiently rigid to allow easy insertion of a breathing apparatus 30 into a patient's mouth with engagement surfaces 35, 36 being positioned relative to the patient's teeth 20, 21. Engagement surfaces 35, 36 should be sized and spaced to easily accommodate teeth 20, 21 in space 38 as the mouthpiece 34 is inserted in the patient's mouth. Space 38 can be configured to require teeth 20, 21 to be substantially aligned as illustrated in FIG. 2 and FIG. 8, or can be sufficiently wide to allow upper and lower jaws 18, 15 to assume an “at rest” position with upper jaw teeth 20 spaced outwardly of lower jaw teeth 21. Likewise, engagement surfaces 35, 36 should be sufficiently flexible to allow engagement surfaces 35, 36 to flex to accommodate the configuration of a patient's upper and lower jaws 18, 15 without irritating either upper and lower lips 24, 26 or the gums, not shown, surrounding the base of teeth 20, 21.

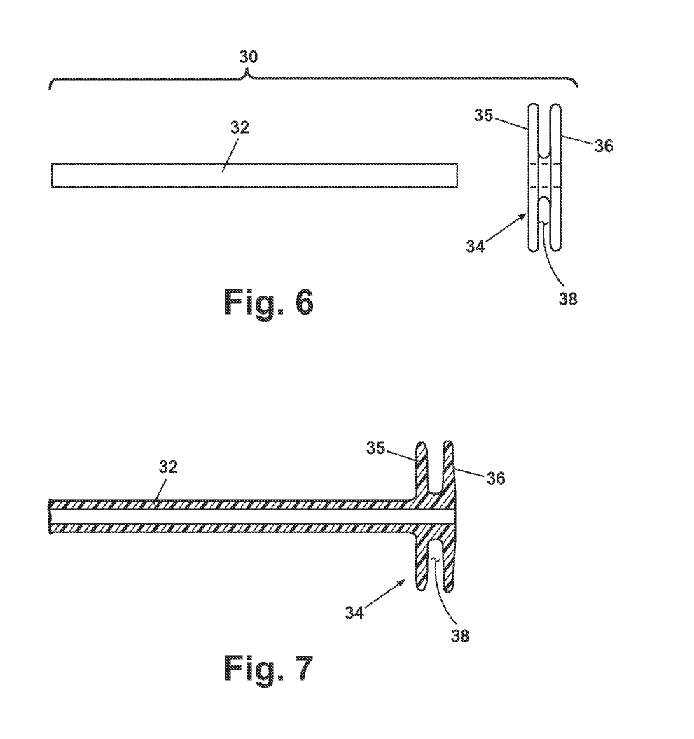

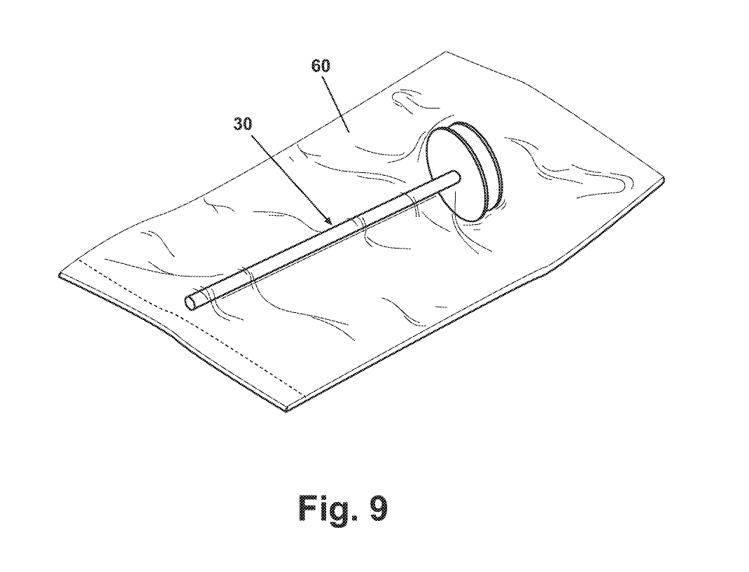

As noted above, breathing apparatus 30 can be molded in two pieces, a breathing tube 32 and a mouthpiece 34, as illustrated in FIG. 6 and assembled by inserting breathing tube 32 into mouthpiece 34. Breathing tube 32 can extend substantially through mouthpiece 34 as illustrated in FIG. 3A. If desired, breathing tube 32 can extend beyond the surface of second engagement surface 36, or can extend only partially through mouthpiece 34. Mouthpiece 34 can be sized to be retained on the end of breathing tube 32 by friction, or, if desired, can be affixed to the end of breathing tube 32 by known methods that can include plastic welding and bonding depending on the materials used for the breathing tube 32 and the mouthpiece 34. Alternately, as illustrated in FIG. 7, breathing apparatus 30 can be molded in one piece, or mouthpiece 34 can be molded on an end of breathing tube 32 to form a one piece breathing apparatus 30. Preferably, breathing apparatus 30 can be packaged in a sterile, hygienic package 60 (see FIG. 9) intended for a single use, and sold in bulk to providers of facial treatments employing materials that can be annoying, irritating or dangerous for their patients. Those skilled in the art will appreciate that, if desired, a breathing apparatus according to the invention can be formed of materials so that the breathing apparatus can be cleaned, sterilized and re-used. However, since the amount of material required and cost to form a breathing apparatus 30 according to the invention is small, it is likely that it will be more economic to fabricate, package and distribute the breathing apparatus according to the invention as a single use product since the cost in time and effort to clean, sterilize and store the cleaned and sterilized breathing apparatus 30 would be greater than the cost of a single use, disposable breathing apparatus 30.

Use of a breathing apparatus 30 according to the invention is quite simple and straightforward. A skin care professional can simply incorporate a breathing apparatus 30 according to the invention into his or her protocol for administering facial treatments using material that might be merely annoying, irritating, distressing or dangerous for patients with asthma or respiratory problems or for pregnant women. The skin care professional can easily instruct a patient on the proper positioning and use of a breathing apparatus 30 according to the invention. The skin care professional can remove a breathing apparatus 30 from a sterile package 60 (see FIG. 9) and insert the breathing tube into the patient's mouth in a position for the patient's teeth 20, 21 to engage mouthpiece 34 with teeth 20, 21 closing into space 38 between the first engagement surface 35 and the second engagement surface 36 and with first engagement surface 35 positioned between teeth 20, 21 and upper and lower lips 24, 26 as shown in FIG. 2 and FIG. 8. The patient need only close their upper and lower jaws 18, 15 so that teeth 20, 21 engage mouthpiece 34 with breathing tube 32 sticking substantially straight out of the patient's mouth. Upper and lower lips 24, 26 will close around breathing tube 32 as upper and lower jaws 18, 15 close bringing teeth 20, 21 into engagement with mouthpiece 34 in space 38. The patient can easily hold breathing apparatus 30 in their mouth even if the lips 24, 26 become numb during the treatment procedure by merely keeping upper and lower jaws 18, 15 substantially closed on mouthpiece 34. With the breathing apparatus in place and the patient comfortable with breathing through the breathing apparatus, the skin care professional can proceed to administer the facial treatment. As soon as the treatment material is removed the breathing apparatus 30 can be removed from the patient's mouth and disposed of simply. Preferably, a fresh, sterile breathing apparatus will be used for each patient and each treatment. The breathing apparatus according to the invention can provide a comfortable, formfitting mouthpiece with a breathing tube extending far enough that a patient will be breathing air untainted and uncontaminated by treatment material or fumes from the treatment material. Use of a breathing apparatus 30 according to the invention can relax the patient and help eliminate any anxiety that might otherwise accompany a facial skin treatment such as “will I be able to hold my breath long enough”, or “will I have a reaction if I accidentally breathe or ingest treatment materials or fumes”. Those skilled in the art will understand that facial treatment materials can be annoying, irritating or dangerous, particularly to patients with sensitivity to chemical materials or with respiratory problems or for women that are pregnant.

An alternate configuration for a breathing apparatus mouthpiece according to the invention can be seen by referring to FIG. 4 and FIG. 4A. Breathing apparatus 40 can be similar to breathing apparatus 30 and can have a breathing tube 42 and a mouthpiece 44 that can function generally similar to the corresponding elements in breathing apparatus 30 illustrated in FIG. 2, FIG. 3, FIG. 3A and FIG. 8. Mouthpiece 44 can have a first engagement surface 45 and a second engagement surface 46 that can be generally circular in shape, and can be longitudinally spaced to form a space 48 to receive teeth 20, 21 similar to breathing apparatus 30. As in the embodiment of breathing apparatus 30, first and second engagement surfaces 45 and 46 can be can overlay teeth along the upper and lower jaws 18, 15. Engagement surfaces 45, 46 can extend toward the upper and lower jaws 18, 15 so that engagement surfaces 45, 46 can overlay teeth 20, 21 in the upper and lower jaws 18, 15. Thus, engagement surfaces 45, 46 can assist in sealing the breathing apparatus 40 to a patient's mouth, particularly if a patient becomes anxious or if a patient's lips become numb from the treatment material, and help assure that a patient will breathe uncontaminated air as the facial treatment procedure occurs. As noted above with respect to breathing apparatus 30, engagement surfaces 45, 46 should be sufficiently rigid to allow easy insertion of a breathing apparatus 40 into a patient's mouth. Engagement surfaces 45, 46 should be sized and spaced to easily accommodate teeth 20, 21 in space 48 as the mouthpiece 44 is inserted in the patient's mouth. Likewise, engagement surfaces 45, 46 should be sufficiently flexible to allow engagement surfaces 45, 46 to flex to accommodate the configuration of a patient's upper and lower jaws 18, 15 without irritating either upper and lower lips 24, 26 or the gums, not shown, surrounding the base of teeth 20, 21. Breathing apparatus 40 can, similar to breathing apparatus 30, be formed as two separate pieces and assembled, or can be molded as a single element. Breathing tube 42 can have a round cross section, or can have other cross section shapes as described above with respect to breathing tube 32. Preferably, breathing apparatus 40 will be a disposable, single use device that can be packaged in sterile package 60 for easy use and subsequent disposal after a facial treatment is completed. Use of breathing apparatus 40 can be similar to use of breathing apparatus 30 described above.

Another configuration for a breathing apparatus mouthpiece according to the invention can be seen by referring to FIG. 5 and FIG. 5A. Breathing apparatus 50 can be similar to breathing apparatus 30 and can have a breathing tube 52 and a mouthpiece 54 that can function generally similar to the corresponding elements in breathing apparatus 30 illustrated in FIG. 2, FIG. 3, FIG. 3A and FIG. 8. Mouthpiece 54 can have a first engagement surface 55 and a second engagement surface 56 that can be generally circular in shape, and can be longitudinally spaced to form a space 58 to receive teeth 20, 21 similar to breathing apparatus 30. A patient's teeth 20, 21 can engage mouthpiece 54 at space 58 that can be a depression or recess in generally cylindrical mouthpiece 54. Engagement surfaces 55, 56 can extend toward the upper and lower jaws 18, 15 so that engagement surfaces 55, 56 can at least partially overlay teeth 20, 21 in the upper and lower jaws 18, 15. Thus, engagement surfaces 55, 56 can assist in retaining breathing apparatus 50 in a patient's mouth, particularly if a patient becomes anxious or if a patient's lips become numb from the treatment material, and help assure that a patient will breathe uncontaminated air as the facial procedure occurs. Mouthpiece 54 can be formed of a soft plastic material with engagement surfaces 55, 56 formed to define space 58 for teeth 20, 21, or can be a generally cylindrical surface with the patient's teeth forming space 58 and engagement surfaces 55, 56 when the breathing apparatus is inserted into the patient's mouth and the patient closes his or her jaws 18, 15 on mouthpiece 54. Instead of soft plastic material, mouthpiece 54 could be formed of wax or other soft material that is compatible with human use, will remain intact through a facial treatment procedure and is resistant to the treatment material. Mouthpiece 54 and engagement surfaces 55, 56, if preformed, should be sized and spaced to easily accommodate teeth 20, 21 in space 58 when the mouthpiece 54 is inserted in the patient's mouth. Likewise, mouthpiece 54 and engagement surfaces 55, 56 should be sufficiently flexible to allow engagement surfaces 55, 56 to flex to accommodate the configuration of a patient's upper and lower jaws 18, 15 without irritating either upper and lower lips 24, 26 or the gums, not shown, surrounding the base of teeth 20, 21. Breathing apparatus 50 can, similar to breathing apparatus 30, be formed as two separate pieces and assembled, can be molded as a single element, or can have a mouthpiece formed on an end of breathing tube 52 if a wax like material is used to form mouthpiece 54. Breathing tube 52 can have a round cross section, or as described above with respect to breathing tube 32, can have other cross sectional shapes as desired. Preferably, breathing apparatus 50 will be a disposable, single use device that can be packaged in sterile package 60 for easy use and subsequent disposal after a facial treatment is completed. Use of breathing apparatus 50 can be similar to use of breathing apparatus 30, 40 described above.

While the invention has been specifically described in connection with certain specific embodiments thereof, it is to be understood that this is by way of illustration and not of limitation, and the scope of the appended claims should be construed as broadly as the prior art will permit.

1. A facial skin treatment procedure including application of a treatment material for exposed facial tissue of a substantially reclined person comprising:
inserting the first end of a breathing tube having a first end and a second end into the person's mouth between the lips and teeth of the person's upper and lower jaws with the second end spaced from person's face for access to air substantially uncontaminated by the treatment material or by fumes of the treatment material;
positioning an engagement surface on the first end between the person's teeth and lips with the first end positioned in the person's substantially closed mouth;
directing the person to close their jaws with the teeth engaging the first end of the breathing tube with the lips overlying the engagement surface thereby maintaining facial tissue exposed for application of treatment material and treatment;
applying facial treatment material to the person's facial tissue;
administering the facial treatment;
removing the facial treatment material from the person's facial tissue; and
removing the breathing tube.

2. The facial skin treatment procedure of claim 1, wherein the step of positioning the engagement surface includes positioning the engagement surface in contact with the person's upper and lower jaw teeth between the person's teeth and lips with the person's lips overlying the engagement surface.

3. The facial skin treatment procedure of claim 1, wherein the engagement surface extends radially outward from the breathing tube.

4. The facial skin treatment procedure of claim 1, wherein the first end further comprises an extended portion arranged to extend between the teeth of the person's upper and lower jaws, and the step of positioning the engagement surface further comprises positioning the extended portion between the person's upper and lower jaws with the engagement surface between the person's teeth and lips to allow the person to grip the breathing tube with their teeth.

5. The facial skin treatment procedure of claim 1, wherein the first end comprises longitudinally spaced engagement surfaces for contact with the person's upper and lower jaw teeth, and the step of positioning the engagement surface comprises positioning one of the engagement surfaces between the lips and the outer surface of the person's teeth and the other of the engagement surfaces adjacent the inside surface of the person's teeth to allow the person to grip the breathing tube with their teeth.
